(19) United States
(12) Patent Application Publication
(10) Pub. No.: US 2020/0289431 A1
(43) Pub. Date: Sep. 17, 2020

(54) COMBINATION OF CANNABIDIOL AND NSAIDS FOR THE TREATMENT OF SCIATICA AND OTHER NERVE PAIN

(71) Applicant: Kristine KuusReichel, San Diego, CA (US)

(72) Inventor: Kristine KuusReichel, San Diego, CA (US)

(21) Appl. No.: 16/814,377

(22) Filed: Mar. 10, 2020

Related U.S. Application Data
(60) Provisional application No. 62/816,737, filed on Mar. 11, 2019.

(51) Int. Cl. A61K 31/05; A61K 31/192

(52) U.S. Cl. CPC A61K 31/05; A61K 31/192

(57) ABSTRACT

Compositions comprising Cannabidiol in combination with non-steroidal anti-inflammatory drugs (NSAIDs) are provided for treating conditions such as sciatica and other nerve pain.

### INCORPORATION BY REFERENCE TO RELATED APPLICATIONS

Any and all priority claims identified in the Application Data Sheet, or any correction thereto, are hereby incorporated by reference under 37 CFR 1.57. This application claims the benefit of U.S. Provisional Application No. 62/816,737, filed Mar. 11, 2019. The aforementioned application is incorporated by reference herein in its entirety, and is hereby expressly made a part of this specification.

### FIELD OF THE INVENTION

Compositions comprising cannabidiol in combination with non-steroidal anti-inflammatory drugs (NSAIDs) are provided for treating conditions such as sciatica and other nerve pain.

### BACKGROUND OF THE INVENTION

Sciatica refers to pain that radiates along the path of the sciatic nerve, which branches from the lower back through the hips and buttocks and down each leg. Typically, sciatica affects only one side of the body. Risk factors for sciatica include: 1) Age—Age-related changes in the spine, such as herniated disks and bone spurs, are the most common causes of sciatica; 2) Obesity—By increasing the stress on the spine, excess body weight can contribute to the spinal changes that trigger sciatica; 3) Occupation—A job that requires twisting the back, carrying heavy loads or driving a motor vehicle for long periods might play a role in sciatica; 4) Prolonged sitting—People who sit for prolonged periods or have a sedentary lifestyle are more likely to develop sciatica than active people are; 5) Diabetes—This condition, which affects the way the body uses blood sugar, increases the risk of nerve damage.

Sciatica most commonly occurs when a herniated disk, bone spur on the spine or narrowing of the spine (spinal stenosis) compresses part of the nerve. This causes inflammation, pain and often some numbness in the affected leg. Although the pain associated with sciatica can be severe, most cases resolve with non-operative treatments in a few weeks. People who have severe sciatica that is associated with significant leg weakness or bowel or bladder changes might be candidates for surgery. Pain that radiates from the lower (lumbar) spine to the buttock and down the back of the leg is the hallmark of sciatica. The discomfort can be felt almost anywhere along the nerve pathway, but it's especially likely to follow a path from the low hack to the buttock and the back of the thigh and calf. The pain can vary widely, from a mild ache to a sharp, burning sensation or excruciating pain. Sometimes it can feel like a jolt or electric shock. It can be worse when one coughs or sneezes, and prolonged sitting can aggravate symptoms. Usually only one side of the body is affected. Some people also have numbness, tingling or muscle weakness in the affected leg or foot. One might have pain in one part of the leg and numbness in another part.

Mild sciatica usually goes away over time. However, in certain cases the symptoms or pain can last longer than a week, can be severe or becomes progressively worse. Although most people recover fully from sciatica, often without treatment, sciatica can potentially cause permanent nerve damage, including loss of feeling in the affected leg, weakness in the affected leg, or loss of bowel or bladder function

### SUMMARY OF THE INVENTION

Treatments that are effective for the treatment of sciatica, either mild sciatica or more severe sciatica, or other nerve pain, are desirable. Accordingly, compositions for treatment of sciatica or nerve pain are provided. Methods are provided for administering the compositions.

Accordingly, in a generally applicable first aspect (i.e., independently combinable with any of the aspects or embodiments identified herein), a method of treatment of sciatica or nerve pain is provided, comprising administering to a patient in need thereof cannabidiol in combination with a non-steroidal anti-inflammatory drug.

In an embodiment of the first aspect, the active ingredients are provided in a unit dosage form.

In an embodiment of the first aspect, the active ingredients are provided in separate unit dosage forms.

In an embodiment of the first aspect, the unit dosage form comprises 15-30 mg cannabidiol is administered.

In an embodiment of the first aspect, the unit dosage form comprises 400-600 mg NSAID is administered.

Accordingly, in a generally applicable second aspect (i.e., independently combinable with any of the aspects or embodiments identified herein), a pharmaceutical composition comprising cannabidiol in combination with a non-steroidal anti-inflammatory drug is provided.

In an embodiment of the second aspect, 15-30 mg cannabidiol is administered.

In an embodiment of the second aspect, 400-600 mg NSAID is administered.

In an embodiment of the second aspect, administering is administering every 6-8 hours or until relief from pain is achieved.

In a generally applicable third aspect (i.e., independently combinable with any of the aspects or embodiments identified herein), a method of treatment of a nerve pain is provided, comprising: administering, to a patient in need thereof, an effective amount of cannabidiol and an effective amount of a non-steroidal anti-inflammatory drug (NSAID).

In an embodiment of the third aspect, the cannabidiol and the NSAID are combined in a pharmaceutical composition in a same unit dosage form.

In an embodiment of the third aspect, the cannabidiol and the NSAID are administered in separate unit dosage forms.

In an embodiment of the third aspect, the separate unit dosage forms are administered at a same time.

In an embodiment of the third aspect, 15-30 mg cannabidiol is administered per dose.

In an embodiment of the third aspect, 400-600 mg NSAID is administered per dose.

In an embodiment of the third aspect, 15-30 mg cannabidiol is administered per dose and wherein 400-600 mg NSAID is administered per dose.

In an embodiment of the third aspect, the NSAID is naproxen sodium.

In an embodiment of the third aspect, the cannabidiol and the NSAID are administered once every 6-8 hours or until relief from pain is achieved.

In an embodiment of the third aspect, the cannabidiol and the NSAID are administered once every 6-8 hours for 2-3 days.

In an embodiment of the third aspect, the nerve pain is sciatica.

In an embodiment of the third aspect, the sciatica is periodic sciatica.

In a generally applicable fourth aspect (i.e., independently combinable with any of the aspects or embodiments identified herein), a method of treatment of periodic sciatica is provided, comprising: administering, to a patient in need thereof, a synergistic combination of 15 mg cannabidiol and 440 mg naproxen sodium.

In an embodiment of the fourth aspect, the synergistic combination is administered at least once, whereby relief from sciatica pain is achieved.

In an embodiment of the fourth aspect, the synergistic combination is administered once, whereby relief from sciatica pain is achieved.

In an embodiment of the fourth aspect, the synergistic combination is administered once every 6-8 hours for 2-3 days, whereby relief from sciatica pain is achieved.

Any of the features of an embodiment of aspects is applicable to all other aspects and embodiments identified herein. Moreover, any of the features of an embodiment is independently combinable, partly or wholly with other embodiments described herein in any way, e.g., one, two, or three or more embodiments may be combinable in whole or in part. Further, any of the features of an embodiment may be made optional to other aspects or embodiments. Any aspect or embodiment of a method can be performed using a composition of another aspect or embodiment, and any aspect or embodiment of a composition can be adapted to be used in a method of another aspect or embodiment.

### DETAILED DESCRIPTION

### Cannabidiol

Cannabidiol (CBD) is a phytocannabinoid discovered in 1940. It is one of some 113 identified cannabinoids in cannabis plants, accounting for up to 40% of the plant's extract. CBD is found in all cannabis species including but not limited to Cannabis satvia (Hemp), Cannabis indica ssp. afganica (Indica), and Cannabis indica ssp. indica (Satvia). Cannabidiol can be taken into the body in multiple ways, including by inhalation of cannabis smoke or vapor, as an aerosol spray into the cheek, and by mouth. It may be supplied as CBD oil containing only CBD as the active ingredient (no added tetrahydrocannabinol (THC) or terpenes), a full-plant CBD-dominant hemp extract oil, capsules, dried cannabis, or as a prescription liquid solution. CBD does not have the same psychoactivity as THC, and may affect the actions of THC. Although in vitro studies indicate CBD may interact with different biological targets, including cannabinoid receptors and other neurotransmitter receptors, the mechanism of action for its biological effects has not been determined.

Cannabidiol has very low affinity for the cannabinoid CB₁ and CB₂ receptors but is said to act as an indirect antagonist of these receptors. At the same time, it may potentiate the effects of THC by increasing CB₁ receptor density or through another CB₁ receptor-related mechanism. Cannabidiol has been found to act as an antagonist of GPR55, a G protein-coupled receptor and putative cannabinoid receptor that is expressed in the caudate nucleus and putamen in the brain. It has also been found to act as an inverse agonist of GPR3, GPR6, and GPR12. Although currently classified as orphan receptors, these receptors are most closely related phylogenetically to the cannabinoid receptors. In addition to orphan receptors, CBD has been shown to act as a serotonin 5-HT_{1A} receptor partial agonist, and this action may be involved in its antidepressant, anxiolytic, and neuroprotective effects. It is an allosteric modulator of the μ- and δ-opioid receptors as well. The pharmacological effects of CBD have additionally been attributed to PPARγ agonism and intracellular calcium release. Research suggests that CBD may exert some of its pharmacological action through its inhibition of fatty acid amide hydrolase (FAAH), which may in turn increase the levels of endocannabinoids, such as anandamide, produced by the body. It has also been speculated that some of the metabolites of CBD have pharmacological effects that contribute to the biological activity of CBD.

The oral bioavailability of CBD is 13 to 19%, while its bioavailability via inhalation is 11 to 45% (mean 31%). The elimination half-life of CBD is 18-32 hours.

Cannabidiol is metabolized in the liver as well as in the intestines by CYP2C19 and CYP3A4 enzymes, and UGT1A7, UGT1A9, and UGT2B7 isoforms.

Compounds related to Cannabidiol include THC (Δ⁹-tetrahydrocannabinol), CBGA (Cannabigerolic acid), THCA (Δ⁹-tetrahydrocannabinolic acid), CBDA (Cannabidiolic acid), CBCA (Cannabichromenenic acid), CBGVA (Cannabigerovarinic acid), THCVA (Tetrahydrocanabivarinic acid), CBDVA (Cannabidivarinic acid), and CBCVA (Cannabichromevarinic acid). While cannabidiol is employed in the methods of treatment and compositions described herein, the aforementioned compounds may be substituted for cannabidiol, or may be employed in conjunction with cannabidiol.

### Non-Steroidal Anti-Inflammatory Drugs (NSAIDs)

Nonsteroidal anti-inflammatory drugs (NSAIDs) are a drug class that reduce pain, decrease fever, prevent blood clots and, in higher doses, decrease inflammation. Side effects depend on the specific drug, but largely include an increased risk of gastrointestinal ulcers and bleeds, heart attack and kidney disease.

NSAIDs work by inhibiting the activity of cyclooxygenase enzymes (COX-1 and/or COX-2). In cells, these enzymes are involved in the synthesis of key biological mediators, namely prostaglandins which are involved in inflammation, and thromboxanes which are involved in blood clotting.

There are two types of NSAID available: non-selective and COX-2 selective. Most NSAIDs are non-selective, and inhibit the activity of both COX-1 and COX-2. These NSAIDs, while reducing inflammation, also inhibit platelet aggregation (especially aspirin) and increase the risk of gastrointestinal ulcers/bleeds. COX-2 selective inhibitors have less gastrointestinal side effects, but promote thrombosis and substantially increase the risk of heart attack. As a result, COX-2 selective inhibitors are generally contraindicated due to the high risk of undiagnosed vascular disease. These differential effects are due to the different roles and tissue localizations of each COX isoenzyme. By inhibiting physiological COX activity, all NSAIDs increase the risk of kidney disease and, through a related mechanism, heart attack.

The most prominent NSAIDs are aspirin, ibuprofen and naproxen, all available over the counter in most countries. Paracetamol (acetaminophen) is generally not considered an NSAID because it has only minor anti-inflammatory activity. It treats pain mainly by blocking COX-2 mostly in the central nervous system, but not much in the rest of the body. Other NSAIDs include but are not limited to celecoxib, diclofenac, diflunisal, etodolac, indomethacin, ketoprofen, ketorolac, nabumetone, oxaprozin, piroxicam, salsalate, sulindac, and tolmetin.

### Neuropathic Pain

Neuropathic pain (neuropathy or nerve pain) is pain caused by damage or disease affecting the somatosensory nervous system. Neuropathic pain may be associated with abnormal sensations called dysesthesia or pain from normally non-painful stimuli (allodynia). It may have continuous and/or episodic (paroxysmal) components. The latter resemble stabbings or electric shocks. Common qualities include burning or coldness, “pins and needles” sensations, numbness and itching.

Neuropathic pain may result from disorders of the peripheral nervous system or the central nervous system (brain and spinal cord). Thus, neuropathic pain may be divided into peripheral neuropathic pain, central neuropathic pain, or mixed (peripheral and central) neuropathic pain.

Central neuropathic pain is found in spinal cord injury, multiple sclerosis, and some strokes. Aside from diabetes (see diabetic neuropathy) and other metabolic conditions, the common causes of painful peripheral neuropathies are herpes zoster infection, HIV-related neuropathies, nutritional deficiencies, toxins, remote manifestations of malignancies, immune mediated disorders and physical trauma to a nerve trunk. Neuropathic pain is common in cancer as a direct result of cancer on peripheral nerves (e.g., compression by a tumor), or as a side effect of chemotherapy (chemotherapy-induced peripheral neuropathy), radiation injury or surgery.

After a peripheral nerve lesion, aberrant regeneration may occur. Neurons become unusually sensitive and develop spontaneously pathological activity and abnormal excitability. This phenomenon is called “peripheral sensitization”.

The (spinal cord) dorsal horn neurons give rise to the spinothalamic tract (STT), which constitutes the major ascending nociceptive pathway. As a consequence of spontaneous activity arising in the periphery, STT neurons develop increased background activity, enlarged receptive fields and increased responses to afferent impulses, including normally innocuous tactile stimuli. This phenomenon is called central sensitization. Central sensitization is an important mechanism of persistent neuropathic pain.

Other mechanisms may take place at the central level after peripheral nerve damage. The loss of afferent signals induces functional changes in dorsal horn neurons. A decrease in the large fiber input decreases the activity of interneurons inhibiting nociceptive neurons i.e. loss of afferent inhibition. Hypoactivity of the descending antinociceptive systems or loss of descending inhibition may be another factor. With the loss of neuronal input (deafferentation) the STT neurons begin to fire spontaneously, a phenomenon designated “deafferentation hypersensitivity.”

Neuroglia (“glial cells”) may play a role in central sensitization. Peripheral nerve injury induces glia to release proinflammatory cytokines and glutamate—which, in turn influence neurons.

Neuropathic pain can be dependent on changes at the cellular and molecular levels. Altered expression of ion channels, changes in neurotransmitters and their receptors as well as altered gene expression in response to neural input are at play. Neuropathic pain is associated with changes in sodium and calcium channel subunit expression resulting in functional changes. In chronic nerve injury, there is redistribution and alteration of subunit compositions of sodium and calcium channels resulting in spontaneous firing at ectopic sites along the sensory pathway.

Neuropathy can result from any type of pain that compresses or impinges on a nerve. A herniated disc, for example, could press against a nearby nerve, causing pain. Neuropathic pain originating from the back or spine may include: chronic pain radiating down the leg (lumbar radiculopathy, or sciatica); chronic pain radiating down the arm (cervical radiculopathy); pain following back surgery that starts gradually and persists, commonly called failed back surgery syndrome.

Other conditions where nerve pain is common include autoimmune diseases, arthritis, cancer, compression/trauma, diabetes, drug side effects, toxic substances, motor neuron diseases, nutritional deficiencies and infectious disease.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, and phosphates.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, fatty acids, fatty alcohols, sterol and sterol derivatives, tocopherols, carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood. When a tincture or other liquid form containing CBD is prepared, animal, vegetable oils, or mineral oils suitable for human consumption can advantageously be employed as diluents. For example, suitable vegetable oils include but are not limited to olive oil, coconut oil, MCT (mixed chain triglicerides derived from coconut oil), and avocado oil.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles, and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

Where a range of values is provided, it is understood that the upper and lower limit, and any intervening value between the upper and lower limit of the range is included.

Any percentages, ratios or other quantities referred to herein are on a weight basis, unless otherwise indicated.

### Pharmaceutical Compositions

Formulations including Cannabidiol and a non-steroidal anti-inflammatory drug are provided, optionally with least one excipient are provided. It is generally preferred to administer the two components of the formulation in a single unit dosage form for oral administration; however, other routes of administration are also contemplated. For example, the Cannabidiol and the NSAID can be administered in separate unit dosage forms. The Cannabidiol can be administered before, at the same time, or after administration of the NSAID. The NSAID can be administered in oral unit dosage form, and the Cannabidiol can be administered in oral unit dosage form, or through inhalation (e.g., as a vapor, a mist, or smoke).

The compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The compositions disclosed herein may be manufactured by a process that is itself known, e.g., by means of conventional mixing, dissolving, granulating, dragee-making, levigating, emulsifying, encapsulating, entrapping, tableting, or extracting processes. Many of the compounds used in the pharmaceutical combinations disclosed herein may be provided as salts with pharmaceutically acceptable counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, the cannabidiol and/or the NSAID component may be combined as active ingredient(s) in intimate admixture with a pharmaceutical carrier according to conventional pharmaceutical compounding techniques. The carrier can take a wide variety of forms depending on the form of preparation desired for administration. Thus, the compositions provided herein can be presented as discrete units suitable for oral administration such as capsules, cachets or tablets each containing a predetermined amount of the active ingredient. Further, the compositions can be presented as an oil, a powder, as granules, as a solution, as a suspension in an aqueous liquid, as a non-aqueous liquid, as an oil-in-water emulsion, or as a water-in-oil liquid emulsion. In addition to the common dosage forms set out above, the compounds provided herein, or pharmaceutically acceptable salts or derivatives thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical compositions may contain the Cannabidiol and/or the NSAID in an amount effective for the desired therapeutic effect. In some embodiments, the compositions are in a unit dosage form and comprise from about 0.1 mg or less to about 5000 mg or more of each active ingredient per unit dosage form. In further embodiments, the compositions comprise from about 1 to about 500 mg per unit dosage form or from about 500 to 5000 mg per unit dosage form. Such dosage forms may be solid, semisolid, liquid, an emulsion, or adapted for delivery via aerosol or the like for inhalation administration.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

Compositions provided herein can be prepared as solutions or suspensions of the active compound(s) in water or oil or other liquid carrier. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Compositions containing a compound provided herein, or pharmaceutically acceptable salt or derivative thereof, can also be prepared in powder or liquid concentrate form for dilution.

Contemplated herein are compositions including Cannabidiol and an NSAID. The Cannabidiol and/or the NSAID may be present in a single formulation or in multiple formulations provided together, or may be unformulated. In some embodiments, Cannabidiol and/or the NSAID can be administered with one or more additional agents together in a single composition. For example, Cannabidiol can be administered in one composition, and at least one NSAID can be administered in a second composition. In a further embodiment, Cannabidiol and the NSAID are co-packaged in a kit. For example, a drug manufacturer, a drug reseller, a physician, a compounding shop, or a pharmacist can provide a kit comprising Cannabidiol and the NSAID for delivery to a patient.

Some embodiments described herein relate to a composition, which can include a therapeutically effective amount of Cannabidiol and the NSAID. The pharmaceutical composition can include Cannabidiol in, for example, >1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition. In some embodiments, the pharmaceutical composition can include a one or more NSAIDS in, for example, >1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition.

Foodstuffs and other comestibles including Cannabidiol and/or an NSAID are provided. Cannabidiol and the NSAID may be added to foodstuffs for consumption by a subject. Cannabidiol and the NSAID may be integrated into one or more ingredients of a foodstuff. Cannabidiol and the NSAID may be prepared as an ingredient, or may be unprepared. Cannabidiol and the NSAID may be added prior to preparation, during preparation, or following preparation. Preparation may without limitation include cooking, mixing, flavoring, seasoning, blending, boiling, frying, baking, or other processes known in the art. Fortification is preferably at a level so as to provide a therapeutic dosage of the Cannabidiol and the NSAID, as described elsewhere herein; however, beneficial effects may also be obtained at amounts below such dosages.

### Indications

Provided herein are compositions and methods for treating conditions including neuropathic pain (neuropathy), including sciatica. These conditions are treated by administration of a combination of cannabidiol in combination with an NSAID.

As will be readily apparent to one skilled in the art, the useful in vivo dosage to be administered and the particular mode of administration will vary depending upon the age, weight, the severity of the condition, and mammalian species treated, the particular forms of the components employed, and the specific use for which these components are employed. The determination of effective dosage levels, that is the dosage levels necessary to achieve the desired result, can be accomplished by one skilled in the art using routine methods, for example, in vivo studies. Reference may be made to, for example, “Estimating the Maximum Safe Starting Dose in Initial Clinical Trials for Therapeutics in Adult Healthy Volunteers,” U.S. Food and Drug Administration, July 2005.

In some embodiments, a method provided herein may comprise administering a therapeutically effective amount of a composition provided herein. In some embodiments, a therapeutically effective amount may be determined by reference to the modulation of a marker of pain (a behavioral marker or self-reported condition of pain level and type).

The dosage may vary broadly, depending upon the desired effects and the therapeutic indication. Alternatively, dosages may be based and calculated upon the surface area or weight of the patient, as understood by those of skill in the art. The exact dosage will be determined on a case-by-case basis, or, in some cases, will be left to the informed discretion of the subject. The daily dosage regimen for an adult human patient may be, for example, an oral dose of the Cannabidiol of from about 0.01 mg to about 10000 mg, from about 1 mg to about 5000 mg, from about 5 mg to about 2000 mg, from about 10 mg to about 1000 mg, or from about 50 mg to about 500 mg. A single dose may include Cannabidiol in about 0.01 mg, about 0.1 mg, about 1 mg, about 5 mg, about 10 mg, about 20 mg, about 50 mg, about 100 mg, about 200 mg, about 300 mg, about 400 mg, about 500 mg, about 600 mg, about 800 mg, about 900 mg, about 1000 mg, about 2000 mg, about 5000 mg, or more. The dosage may be adjusted according to the body mass of the subject, for example, the dosage may be about 0.001 mg/kg, about 0.01 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, or higher. It is generally preferred to administer a dose of 15 to 30 mg of Cannabidiol.

The daily dosage regimen for an adult human patient may be, for example, an oral dose of the NSAID of from about 0.01 mg to about 10000 mg, from about 1 mg to about 5000 mg, from about 5 mg to about 2000 mg, from about 10 mg to about 1000 mg, or from about 50 mg to about 500 mg. A single dose may include one or more NSAIDs in about 0.01 mg, about 0.1 mg, about 1 mg, about 5 mg, about 10 mg, about 20 mg, about 50 mg, about 100 mg, about 200 mg, about 300 mg, about 400 mg, about 500 mg, about 600 mg, about 800 mg, about 900 mg, about 1000 mg, about 2000 mg, about 5000 mg, or more. The dosage may be adjusted according to the body mass of the subject, for example, the dosage may be about 0.001 mg/kg, about 0.01 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, or higher. It is generally preferred to administer 440 mg of the NSAID by mouth the first hour as needed, and then 220 mg by mouth every eight to 12 hours, again, as needed, with a maximum dosage not exceeding 440 mg in any eight- to 12-hour period, or 660 mg in any 24-hour period.

The dosage may be a single one or a series of two or more given in the course of one or more days, as is appropriate for the individual subject. In some embodiments, the compounds will be administered until symptoms subside, or for a period of continuous therapy, for example for about a week or more (e.g., one week, two weeks, three weeks, four weeks, five weeks, six weeks, seven weeks, eight weeks, or more), for several weeks, for about a month or more (e.g., one month, two months, three months, four months, five months, six months, seven months, eight months, nine months, ten months, eleven months, twelve months, or more), for about a year or more, or for a plurality of years. In some embodiments, the Cannabidiol can be administered, inhaled, or ingested one time per day, two times per day, three times per day, or more. In some embodiments, the NSAID can be administered or ingested one time per day, two times per day, three times per day, or more. It is generally preferred to administer 15-30 ng Cannabidiol with 440 mg of the NSAID by mouth, with this dosing repeated every 6-8 hours until relief is obtained.

As will be understood by those of skill in the art, in certain situations it may be necessary to administer the compounds disclosed herein in amounts that exceed the above-stated, preferred dosage range in order to effectively treat a subject.

Unit dosage forms can also be provided, e.g., individual packages with a premeasured amount of the composition, configured for administration on a predetermined schedule. Unit dosage forms configured for administration one to three times a day are preferred; however, in certain embodiments it may be desirable to configure the unit dosage form for administration more than three times a day, or less than one time per day.

Dosage amount and interval may be adjusted to the individual subject to provide plasma levels of the active moiety which are sufficient to maintain predetermined parameters, indicators, or marker values, or minimal effective concentration (MEC). Dosages necessary to achieve the desired result will depend on individual characteristics and route of administration. However, assays, for example, HPLC assays or bioassays, may be used to determine serum concentrations.

### EXAMPLES

A patient suffering from periodic sciatica pain used NSAID and rest as a standard treatment. It took 2 to 3 days for the episode to resolve and left the patient homebound.

The patient took two gelcaps (440 mg naproxen sodium) with 15 mg of cannabidiol and observed that the combination therapy synergistically relieved the sciatica episode in a matter of hours, compared to either component administered alone.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method of treatment of a nerve pain, comprising:
administering, to a patient in need thereof, an effective amount of cannabidiol and an effective amount of a non-steroidal anti-inflammatory drug (NSAID).

2. The method of claim 1, wherein the cannabidiol and the NSAID are combined in a pharmaceutical composition in a same unit dosage form.

3. The method of claim 1, wherein the cannabidiol and the NSAID are administered in separate unit dosage forms.

4. The method of claim 1, wherein the separate unit dosage forms are administered at a same time.

5. The method of claim 1, wherein 15-30 mg cannabidiol is administered per dose.

6. The method of claim 1, wherein 400-600 mg NSAID is administered per dose.

7. The method of claim 1, wherein 15-30 mg cannabidiol is administered per dose and wherein 400-600 mg NSAID is administered per dose.

8. The method of claim 1, wherein the NSAID is naproxen sodium.

9. The method of claim 1, wherein the cannabidiol and the NSAID are administered once every 6-8 hours or until relief from pain is achieved.

10. The method of claim 1, wherein the cannabidiol and the NSAID are administered once every 6-8 hours for 2-3 days.

11. The method of claim 1, wherein the nerve pain is sciatica.

12. The method of claim 1, wherein the sciatica is periodic sciatica.

13. A method of treatment of periodic sciatica, comprising:
administering, to a patient in need thereof, a synergistic combination of 15 mg cannabidiol and 440 mg naproxen sodium.

14. The method of claim 13, wherein the synergistic combination is administered at least once, whereby relief from sciatica pain is achieved.

15. The method of claim 13, wherein the synergistic combination is administered once, whereby relief from sciatica pain is achieved.

16. The method of claim 13, wherein the synergistic combination is administered once every 6-8 hours for 2-3 days, whereby relief from sciatica pain is achieved.
